(19) United States
(12) Patent Application Publication
(10) Pub. No.: US 2025/0251379 A1
(43) Pub. Date: Aug. 7, 2025

(54) CARBON DIOXIDE CAPTURE APPARATUS, CARBON DIOXIDE CAPTURE METHOD, AND NON-TRANSITORY COMPUTER-READABLE MEDIUM

(71) Applicant: TOYOTA JIDOSHA KABUSHIKI KAISHA, Toyota-shi (JP)

(72) Inventor: Hiroyuki FUJIKAWA, Iizuka-shi (JP)

(73) Assignee: TOYOTA JIDOSHA KABUSHIKI KAISHA, Toyota-shi (JP)

(21) Appl. No.: 18/987,590

(22) Filed: Dec. 19, 2024

(30) Foreign Application Priority Data
Feb. 6, 2024 (JP) 2024-016329

(51) Int. Cl. G01N 33/00

(52) U.S. Cl. CPC G01N 33/004

(57) ABSTRACT

A carbon dioxide capture apparatus according to the present disclosure includes: a first apparatus including a first adsorbent that adsorbs at least a part of carbon dioxide contained in outside air to be processed; a second apparatus including a second adsorbent that adsorbs at least a part of carbon dioxide contained in the outside air; and an exhaust path that communicates the first apparatus with the second apparatus, in which the outside air that has passed through the first apparatus in such a way that at least a part of carbon dioxide is adsorbed by the first adsorbent passes through the second apparatus via the exhaust path so as to cool the second adsorbent.

### CROSS REFERENCE TO RELATED APPLICATIONS

This application is based upon and claims the benefit of priority from Japanese patent application No. 2024-016329, filed on Feb. 6, 2024, the disclosure of which is incorporated herein in its entirety by reference.

### BACKGROUND

The present disclosure relates to a carbon dioxide capture apparatus, a carbon dioxide capture method, and a carbon dioxide capture program. Patent Literature 1 discloses a carbon dioxide capture apparatus for heating or cooling a laminated carbon dioxide (CO₂) adsorbent by providing a heat medium flow passage that penetrates through the CO₂ adsorbent. The heat medium disclosed in Patent Literature 1 does not directly contact the CO₂ adsorbent.
[Patent Literature 1] Japanese Unexamined Patent Application Publication No. 2023-013167

### SUMMARY

While Patent Literature 1 uses cooling water as means for cooling the CO₂ adsorbent, a new motive power such as a pump is required in order to cause this cooling water to flow, which causes a degradation in CO₂ capture efficiency relative to a required energy. An apparatus capable of improving cooling efficiency has thus been desired.

The present disclosure has been made in order to solve the aforementioned problem, and an object of the present disclosure is to provide a carbon dioxide capture apparatus, a carbon dioxide capture method, and a carbon dioxide capture program capable of improving a cooling efficiency of a CO₂ adsorbent.

A carbon dioxide capture apparatus according to one aspect of the present disclosure includes: a first apparatus including a first adsorbent that adsorbs at least a part of carbon dioxide contained in outside air to be processed; a second apparatus including a second adsorbent that adsorbs at least a part of the carbon dioxide contained in the outside air; and an exhaust path configured to communicate the first apparatus with the second apparatus, in which the outside air that has passed through the first apparatus in such a way that at least a part of the carbon dioxide is adsorbed by the first adsorbent passes through the second apparatus via the exhaust path so as to cool the second adsorbent.

In the above carbon dioxide capture apparatus, the first adsorbent and the second adsorbent may be each disposed inside a tubular cylindrical vessel, the first apparatus may include one or a plurality of the cylindrical vessels including the first adsorbent, the second apparatus may include one or a plurality of the cylindrical vessels including the second adsorbent, at least a part of the carbon dioxide of the outside air that has passed through the inside of the cylindrical vessel may be adsorbed, and the outside air that has passed through an outer periphery of the cylindrical vessel may cool the second adsorbent.

In the above carbon dioxide capture apparatus, the first apparatus may include: a first inflow port through which the outside air passing through the inside of the cylindrical vessel flows in; a first discharge port from which the outside air that has passed through the inside of the cylindrical vessel is discharged; a first introduction port through which the outside air passing through an outer periphery of the cylindrical vessel is introduced; and a first release port from which the outside air that has passed through the outer periphery of the cylindrical vessel is released, and the second apparatus includes: a second inflow port through which the outside air passing through the inside of the cylindrical vessel flows in; a second discharge port through which the outside air that has passed through the inside of the cylindrical vessel is discharged; a second introduction port through which the outside air passing through an outer periphery of the cylindrical vessel is introduced; and a second release port through which the outside air that has passed through an outer periphery of the cylindrical vessel is released.

The above carbon dioxide capture apparatus may further include; a plurality of open or close valves disposed in the first apparatus, the second apparatus, and the exhaust path; and a control unit configured to switch the first state to the second state by controlling the plurality of open or close valves, rising of temperature of the first adsorbent, and rising of temperature of the second adsorbent, in which in the first state, the outside air that has passed through the first apparatus in such a way that at least a part of the carbon dioxide is adsorbed by the first adsorbent may pass through the second apparatus via the exhaust path so as to cool the second adsorbent, and in the second state, the temperature of the first adsorbent may be raised, the carbon dioxide may be caused to be desorbed from the first adsorbent, and the carbon dioxide may be released to the outside.

The above carbon dioxide capture apparatus may further include: a plurality of open or close valves disposed in the first apparatus, the second apparatus, and the exhaust path; and a control unit configured to switch between a first state and a third state by controlling the plurality of open or close valves, rising of temperature of the first adsorbent, and rising of temperature of the second adsorbent, in which in the first state, the outside air that has passed through the first apparatus in such a way that at least a part of the carbon dioxide is adsorbed by the first adsorbent may pass through the second apparatus via the exhaust path so as to cool the second adsorbent, and in the third state, the outside air that has passed through the second apparatus in such a way that at least a part of the carbon dioxide is adsorbed by the second adsorbent may pass through the first apparatus via the exhaust path so as to cool the first adsorbent.

In the above carbon dioxide capture apparatus, after the control unit switches the first state to a second state, it may switch the second state to the third state, after the control unit switches the third state to a fourth state, it may switch the fourth state to the first state, in the second state, the temperature of the first adsorbent may be raised, the carbon dioxide may be caused to be desorbed from the first adsorbent, and the carbon dioxide may be released to the outside, and in the fourth state, the temperature of the second adsorbent may be raised, the carbon dioxide may be caused to be desorbed from the second adsorbent, and the carbon dioxide may be released to the outside.

In the above carbon dioxide capture apparatus, the exhaust path may include: a first exhaust path through which the outside air flows from the first apparatus to the second apparatus; and a second exhaust path through which the outside air flows from the second apparatus to the first apparatus, and the first exhaust path and the second exhaust path may include parts communicated with the first apparatus and the second apparatus from directions different from each other.

In the above carbon dioxide capture apparatus, the first apparatus may include: a first inflow port through which the outside air flows in; a first discharge port through which the outside air flowing in through the first inflow port is discharged; a first introduction port through which the outside air discharged from the second apparatus is introduced; and a first release port through which the outside air introduced from the first introduction port is released to the outside, the second apparatus may include: a second inflow port through which the outside air flows in; a second discharge port through which the outside air flowing in through the second inflow port is discharged; a second introduction port through which the outside air discharged from the first apparatus is introduced; and a second release port through which the outside air introduced from the second introduction port is released to the outside, the exhaust path may include: a common space disposed between the first apparatus and the second apparatus; a first discharge space disposed between the common space and the first discharge port; a first introduction space disposed between the common space and the first introduction port; a second discharge space disposed between the common space and the second discharge port; and a second introduction space disposed between the common space and the second introduction port, the plurality of open or close valves may include: a first inflow valve that opens or closes the first inflow port; a first discharge valve that opens or closes a part between the common space and the first discharge space; a first introduction valve that opens or closes a part between the common space and the first introduction space; a second inflow valve that opens or closes the second inflow port; a second discharge valve that opens or closes a part between the common space and the second discharge space; and a second introduction valve that opens or closes a part between the common space and the second introduction space, the first discharge valve, the first introduction valve, the second discharge valve, and the second introduction valve may be disposed in the exhaust path, the first exhaust path may include the first discharge space, the common space, and the second introduction space that are communicated with each other, and the second exhaust path may include the second discharge space, the common space, and the first introduction space that are communicated with each other.

The above carbon dioxide capture apparatus may further include: a first desorption valve that opens or closes a part between the first discharge space and the outside; and a second desorption valve that opens or closes a part between the second discharge space and the outside.

In the above carbon dioxide capture apparatus, a release port that releases the outside air to the outside may be disposed on the side of the introduction port opposite to the side thereof that communicates with the first exhaust path in the second apparatus, and an inflow port through which the outside air flows in from the outside may be disposed on the side of the discharge port opposite to the side thereof that communicates with the second exhaust path in the second apparatus.

A carbon dioxide capture method according to one aspect of the present disclosure is a carbon dioxide capture method performed using a carbon dioxide capture apparatus including: a first apparatus including a first adsorbent that adsorbs at least a part of carbon dioxide contained in outside air to be processed; a second apparatus including a second adsorbent that adsorbs at least a part of the carbon dioxide contained in the outside air; and an exhaust path configured to communicate the first apparatus with the second apparatus, the carbon dioxide capture method including: a first adsorption process for causing the outside air to be passed through the first apparatus in such a way that at least a part of the carbon dioxide is adsorbed by the first adsorbent; and a first cooling process for causing the outside air to pass through the second apparatus via the exhaust path in such a way that the outside air that has passed through the first apparatus in the first adsorption process cools the second adsorbent.

A carbon dioxide capture program according to one aspect of the present disclosure is a carbon dioxide capture program executed using a carbon dioxide capture apparatus including: a first apparatus including a first adsorbent that adsorbs at least a part of carbon dioxide contained in outside air to be processed; a second apparatus including a second adsorbent that adsorbs at least a part of the carbon dioxide contained in the outside air; and an exhaust path configured to communicate the first apparatus with the second apparatus; a plurality of open or close valves disposed in the first apparatus, the second apparatus, and the exhaust path; and a control unit configured to control the plurality of open or close valves, rising of temperature of the first adsorbent, and rising of temperature of the second adsorbent, the program causing a computer to execute: a first adsorption process for causing the outside air to be passed through the first apparatus in such a way that at least a part of the carbon dioxide is adsorbed by the first adsorbent; and a first cooling process for causing the outside air to pass through the second apparatus via the exhaust path in such a way that the outside air that has passed through the first apparatus in the first adsorption process cools the second adsorbent.

According to the present disclosure, it is possible to provide a carbon dioxide capture apparatus, a carbon dioxide capture method, and a carbon dioxide capture program capable of improving cooling efficiency.

The above and other objects, features and advantages of the present disclosure will become more fully understood from the detailed description given hereinbelow and the accompanying drawings.

### BRIEF DESCRIPTION OF DRAWINGS

FIG. 1 is a cross-sectional view illustrating a carbon dioxide capture apparatus according to a first embodiment, and shows a first process in a carbon dioxide capture apparatus 1;

FIG. 2 is a cross-sectional view illustrating the carbon dioxide capture apparatus according to the first embodiment, and shows a second process in the carbon dioxide capture apparatus 1;

FIG. 3 is a cross-sectional view illustrating the carbon dioxide capture apparatus according to the first embodiment, and shows a third process in the carbon dioxide capture apparatus 1;

FIG. 4 is a cross-sectional view illustrating the carbon dioxide capture apparatus according to the first embodiment, and shows a fourth process in the carbon dioxide capture apparatus 1;

FIG. 5 is a perspective view illustrating a first adsorbent in the carbon dioxide capture apparatus according to the first embodiment;

FIG. 6 is a flowchart illustrating a carbon dioxide capture method using the carbon dioxide capture apparatus according to the first embodiment; and

FIG. 7 is a block diagram illustrating a control unit including a computer in a carbon dioxide capture apparatus according to each of the first and second embodiments.

### DESCRIPTION OF EMBODIMENTS

Hereinafter, with reference to the drawings, specific configurations of the present embodiments will be described. The following description indicates suitable embodiments of the present disclosure and the scope of the present disclosure is not limited to the following embodiments. Further, all the components/structures described in the embodiments are not necessarily indispensable as means for solving the problem. For clarifying the explanation, the following description and drawings are partially omitted and simplified as appropriate. The same reference numerals (or symbols) are assigned to the same elements throughout the drawings and redundant descriptions thereof are omitted as appropriate.

### First Embodiment

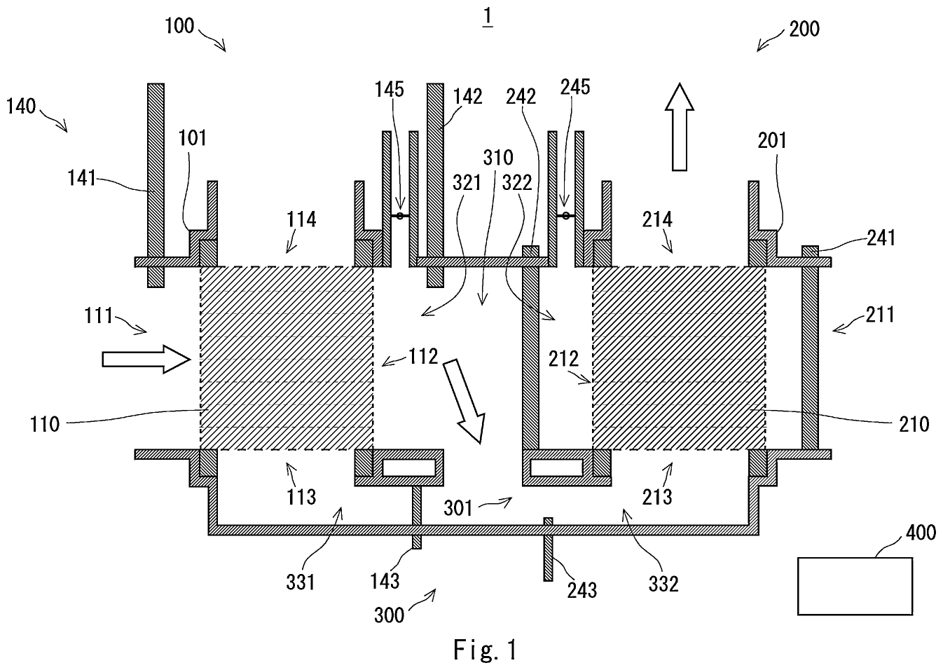

A carbon dioxide capture apparatus and a carbon dioxide capture method according to a first embodiment will be described. FIGS. 1 to 4 are cross-sectional views illustrating a carbon dioxide capture apparatus 1 according to the first embodiment. FIGS. 1 to 4 respectively show a first process, a second process, a third process, and a fourth process in the carbon dioxide capture apparatus 1. The first process includes a first adsorption process and a first cooling process. The second process includes a first temperature raising process and a first desorption process. The third process includes a second adsorption process and a second cooling process. The fourth process includes a second temperature raising process and a second desorption process. The state of the first process is referred to as a first state. The state of the second process is referred to as a second state. The state of the third process is referred to as a third state. The state of the fourth process is referred to as a fourth state. As shown in FIGS. 1 to 4, the carbon dioxide capture apparatus 1 includes a first apparatus 100, a second apparatus 200, an exhaust path 300, a plurality of open or close valves 140, and a control unit 400.

### First Apparatus

The first apparatus 100 includes a first adsorbent 110. The first adsorbent 110 adsorbs at least a part of carbon dioxide contained in outside air to be processed. The first apparatus 100 also includes a housing 101. The housing 101 in the first apparatus 100 includes a first inflow port 111, a first discharge port 112, a first introduction port 113, and a first release port 114.

The first inflow port 111 is an opening through which the outside air to be processed flows in. The first discharge port 112 is an opening through which the outside air flowing from the first inflow port 111 is discharged. The first introduction port 113 is an opening through which the outside air discharged from the second apparatus 200 is introduced via the exhaust path 300. The first release port 114 is an opening through which the outside air introduced from the first introduction port 113 is released to the outside. A fan may be disposed outside the first inflow port 111. Accordingly, it is possible to introduce the outside air to be processed into the first apparatus 100 from the first inflow port 111.

### Second Apparatus

The second apparatus 200 includes a second adsorbent 210. The second adsorbent 210 adsorbs at least a part of carbon dioxide contained in the outside air to be processed. The second apparatus 200 also includes a housing 201. The housing 201 in the second apparatus 200 includes a second inflow port 211, a second discharge port 212, a second introduction port 213, and a second release port 214.

The second inflow port 211 is an opening through which the outside air to be processed flows in. The second discharge port 212 is an opening through which the outside air flowing in through the second inflow port 211 is discharged. The second introduction port 213 is an opening through which the outside air discharged from the first apparatus 100 is introduced via the exhaust path 300. The second release port 214 is an opening through which the outside air introduced from the second introduction port 213 is released to the outside. A fan may be disposed outside the second inflow port 211. Accordingly, the outside air to be processed can be introduced into the second apparatus 200 from the second inflow port 211.

### <Exhaust Path>

The exhaust path 300 communicates the first apparatus 100 with the second apparatus 200. Accordingly, the outside air that has passed through the first apparatus 100 in such a way that at least a part of carbon dioxide contained in the outside air is adsorbed by the first adsorbent 110 passes through the second apparatus 200 via the exhaust path 300 so as to cool the second adsorbent 210. Further, the outside air that has passed through the second apparatus 200 in such a way that at least a part of carbon dioxide contained in the outside air is adsorbed by the second adsorbent 210 passes through the first apparatus 100 via the exhaust path 300 so as to cool the first adsorbent 110.

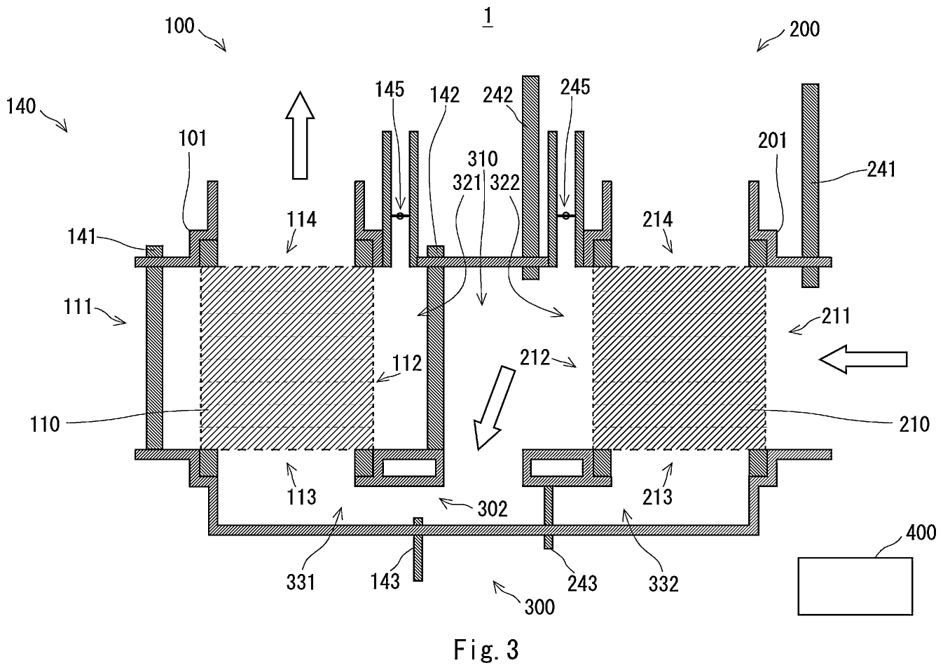

The exhaust path 300 includes a first exhaust path 301 (see FIG. 1) and a second exhaust path 302 (see FIG. 3). The first exhaust path 301 is a path through which the outside air flows from the first apparatus 100 to the second apparatus 200. The second exhaust path 302 is a path through which the outside air flows from the second apparatus 200 to the first apparatus 100. The first exhaust path 301 and the second exhaust path 302 include parts communicated with the first apparatus 100 and the second apparatus 200 from directions different from each other.

The first release port 114 which releases the outside air to the outside is disposed on the side of the first introduction port 113 opposite to the side thereof that communicates with the second exhaust path 302 in the first apparatus 100. The first inflow port 111 through which the outside air flows in from the outside is disposed on the side of the first discharge port 112 opposite to the side thereof that communicates with the first exhaust path 301 in the first apparatus 100. The second release port 214 that releases the outside air to the outside is disposed on the side of the second introduction port 213 opposite to the side thereof that communicates with the first exhaust path 301 in the second apparatus 200. The second inflow port 211 through which the outside air flows in from the outside is disposed on the side of the second discharge port 212 opposite to the side thereof that communicates with the second exhaust path 302 in the second apparatus 200.

Further, the exhaust path 300 includes a common space 310, a first discharge space 321, a first introduction space 331, a second discharge space 322, and a second introduction space 332. The common space 310 is disposed between the first apparatus 100 and the second apparatus 200. The first discharge space 321 is disposed between the common space 310 and the first discharge port 112. The first introduction space 331 is disposed between the common space 310 and the first introduction port 113. The second discharge space 322 is disposed between the common space 310 and the second discharge port 212. The second introduction space 332 is disposed between the common space 310 and the second introduction port 213.

Therefore, the first exhaust path 301 includes the first discharge space 321, the common space 310, and the second introduction space 332. The second exhaust path 302 includes the second discharge space 322, the common space 310, and the first introduction space 331.

### <Open or Close Valves>

The plurality of open or close valves 140 include a first inflow valve 141, a first discharge valve 142, a first introduction valve 143, a first desorption valve 145, a second inflow valve 241, a second discharge valve 242, a second introduction valve 243, and a second desorption valve 245. The first inflow valve 141 opens or closes the first inflow port 111. The first discharge valve 142 opens or closes a part between the common space 310 and the first discharge space 321. The first introduction valve 143 opens or closes a part between the common space 310 and the first introduction space 331. The first desorption valve 145 opens or closes a part between the first discharge space 321 and the outside. The second inflow valve 241 opens or closes the second inflow port 211. The second discharge valve 242 opens or closes a part between the common space 310 and the second discharge space 322. The second introduction valve 243 opens or closes a part between the common space 310 and the second introduction space 332. The second desorption valve 245 opens or closes a part between the second discharge space 322 and the outside.

The plurality of open or close valves 140 are disposed in the first apparatus 100, the second apparatus 200, and the exhaust path 300. In the plurality of open or close valves 140, the first inflow valve 141, the first discharge valve 142, the first introduction valve 143, and the first desorption valve 145 are disposed in the first apparatus 100. In the plurality of open or close valves 140, the second inflow valve 241, the second discharge valve 242, the second introduction valve 243, and the second desorption valve 245 are disposed in the second apparatus 200.

Further, in the plurality of open or close valves 140, the first discharge valve 142, the first introduction valve 143, the second discharge valve 242, and the second introduction valve 243 are disposed in the exhaust path 300. Therefore, the exhaust path 300 includes, of the plurality of open or close valves 140, the first discharge valve 142, the first introduction valve 143, the second discharge valve 242, and the second introduction valve 243.

Then, the first exhaust path 301 includes the first discharge space 321, the common space 310, and the second introduction space 332 that are communicated as a result of opening of the first discharge valve 142 and the second introduction valve 243. In this case, the second discharge valve 242 and the first introduction valve 143 are closed. The second exhaust path 302 includes the second discharge space 322, the common space 310, and the first introduction space 331 communicated as a result of opening of the second discharge valve 242 and the first introduction valve 143. In this case, the first discharge valve 142 and the second introduction valve 243 are closed.

As described above, the plurality of open or close valves 140 are provided in the first exhaust path 301 and the second exhaust path 302. The plurality of open or close valves 140 each include a drive unit so as to be able to open or close a predetermined path.

### <First Adsorbent, Second Adsorbent>

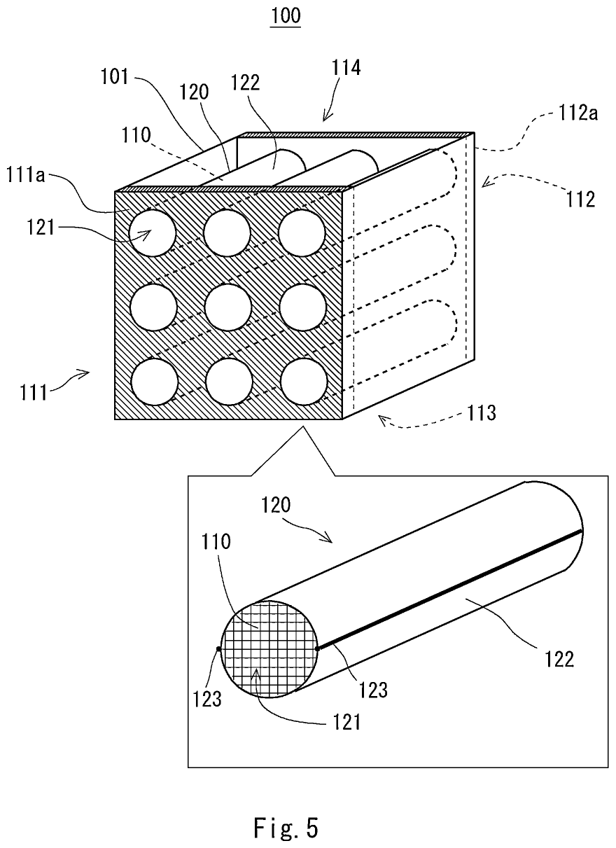

FIG. 5 is a perspective view illustrating the first adsorbent 110 in the carbon dioxide capture apparatus 1 according to the first embodiment. FIG. 5 also shows a diagram in which one cylindrical vessel 120 is enlarged. Further, while the first adsorbent 110 is shown in FIG. 5, the second adsorbent 210 also has a similar configuration. Therefore, the explanation of the first adsorbent 110 in the first apparatus 100 shall be applied to the explanation of the second adsorbent 210 in the second apparatus 200. As shown in FIG. 5, the first adsorbent 110 may be disposed in an inside 121 of the tubular cylindrical vessel 120.

The first apparatus 100 includes one or more cylindrical vessels 120 each including the first adsorbent 110. The second apparatus 200 includes one or more cylindrical vessels 120 each including the second adsorbent 210. At least a part of carbon dioxide of the outside air that has passed through the inside 121 of the cylindrical vessel 120 is adsorbed. The outside air that has passed through an outer periphery 122 of the cylindrical vessel 120 cools the first adsorbent 110 and the second adsorbent 210.

An outer peripheral surface of the cylindrical vessel 120 may be formed of a coating member in such a way that the inside 121 and the outer periphery 122 are isolated from each other. The coating member may contain, for example, a resin, metal, or the like. In order to increase an area where the outside air that has passed through the outer periphery 122 contacts the cylindrical vessel 120 and to improve the cooling performance, a fin, a shotblast or the like may be provided on the outer peripheral surface.

A plurality of tubular cylindrical vessels 120 are disposed inside the housing 101 of the first apparatus 100. One cylindrical vessel 120 may instead be disposed inside the housing 101 of the first apparatus 100. It is assumed, in the following description, that the plurality of cylindrical vessels 120 are disposed in the first apparatus 100. The plurality of tubular cylindrical vessels 120 are disposed between the first inflow port 111 and the first discharge port 112. For example, the plurality of cylindrical vessels 120 are disposed in such a way that the central axis of the cylindrical vessel 120 is extended from the first inflow port 111 toward the first discharge port 112. A shielding plate 111a may be disposed in the first inflow port 111. One end of each of the cylindrical vessels 120 penetrates through the shielding plate 111a. Further, a shielding plate 112a may be disposed in the first discharge port 112. The other end of each of the cylindrical vessels 120 penetrates through the shielding plate 112a.

According to the aforementioned configuration, the outside air flowing from the first inflow port 111 is able to pass through the inside 121 of the cylindrical vessel 120. On the other hand, the outside air flowing from the first inflow port 111 may not pass through the outer periphery 122 of the cylindrical vessel 120. Further, the outside air introduced from the first introduction port 113 is able to pass through the outer periphery 122 of the cylindrical vessel 120. On the other hand, the outside air introduced from the first introduction port 113 is able not to pass through the inside 121 of the cylindrical vessel 120.

As described above, the first inflow port 111 may be an opening through which the outside air passing through inside the cylindrical vessel 120 flows in. The first discharge port 112 may be an opening through which the outside air that has passed through the inside of the cylindrical vessel 120 is discharged. The first introduction port 113 may be an opening through which the outside air passing through the outer periphery of the cylindrical vessel 120 is introduced. The first release port 114 may be an opening through which the outside air that has passed through the outer periphery of the cylindrical vessel 120 is released.

Likewise, the second inflow port 211 may be an opening through which the outside air passing through inside the cylindrical vessel 120 flows in. The second discharge port 212 may be an opening through which the outside air that has passed through the inside of the cylindrical vessel 120 is discharged. The second introduction port 213 may be an opening through which the outside air passing through the outer periphery of the cylindrical vessel 120 is introduced. The second release port 214 may be an opening through which the outside air that has passed through the outer periphery of the cylindrical vessel 120 is released.

The first adsorbent 110 and the second adsorbent 210 may each include, for example, a Direct Air Capture (hereinafter referred to as a DAC) member. A DAC configured to have a honeycomb structure may be included inside the cylindrical vessel 120. It is therefore possible to increase the surface area that the outside air contacts.

The cylindrical vessel 120 may include a heating member 123. The heating member 123 may be an electrode that applies current to the first adsorbent 110 and the second adsorbent 210. The heating member 123 raises the temperature of the first adsorbent 110 and that of the second adsorbent 210 by applying the current to the first adsorbent 110 and the second adsorbent 210. The heating member 123 is not limited to be an electrode and may instead be a heater or the like as long as it enables the temperature of the first adsorbent 110 and that of the second adsorbent 210 to be raised. By raising the temperature of the first adsorbent 110 and that of the second adsorbent 210, carbon dioxide adsorbed by the first adsorbent 110 and the second adsorbent 210 can be desorbed.

As described above, the carbon dioxide capture apparatus 1 according to this embodiment may use DAC by temperature swing as the first adsorbent 110 and the second adsorbent 210. In this case, it is required to cool the first adsorbent 110 and the second adsorbent 210 after the temperature is raised.

In general, a method for cooling the first adsorbent 110 and the second adsorbent 210 may include various methods such as water cooling by a water-cooling apparatus or air cooling by an air cooling apparatus. In either case, additional motive power is required. On the other hand, in this embodiment, the outside air that has passed through the first apparatus 100 so as to be adsorbed by the first adsorbent 110 in the inside 121 of the cylindrical vessel 120 passes through the second apparatus 200 so as to cool the outer periphery 122 of the cylindrical vessel 120 including the second adsorbent 210. That is, by exhausting the exhaust air at the time of adsorption via the cylindrical vessel 120 that needs to be cooled, the adsorbent can be cooled without using a new motive power.

Further, since the path of the exhaust air is formed near the cylindrical vessel 120 including the first adsorbent 110 and the second adsorbent 210, it can be insulated by air. Accordingly, it is possible to reduce a heat transfer path from the first adsorbent 110 and the second adsorbent 210 when carbon dioxide is desorbed by raising the temperature of the first adsorbent 110 and that of the second adsorbent 210. It is therefore possible to reduce the heat loss.

### <Control Unit>

The control unit 400 is connected to the plurality of open or close valves 140 in such a way that it can transmit an operation signal to the plurality of open or close valves 140 via at least one of wired or wireless communication line. The control unit 400 thus controls the opening or closing of the plurality of open or close valves 140. Further, the control unit 400 is connected to the heating member 123 in a state in which it can transmit an operation signal to the heating member 123 that raises the temperature of the first adsorbent 110 and that of the second adsorbent 210. Accordingly, the control unit 400 controls the rising of temperature of the first adsorbent 110 and that of the second adsorbent 210. Further, the control unit 400 is connected to the fan in a state in which it can transmit an operation signal to a fan that causes the outside air to flow into the first apparatus 100 and the second apparatus 200. Accordingly, the control unit 400 controls the fan.

The control unit 400 switches the first state to the second state by controlling the plurality of open or close valves 140, the rising of temperature of the first adsorbent 110, and the rising of temperature of the second adsorbent 210. Further, after the control unit 400 switches the first state to the second state, it switches the second state to the third state by controlling the plurality of open or close valves 140, the rising of temperature of the first adsorbent 110, and the rising of temperature of the second adsorbent 210. Then, after the control unit 400 switches the third state to the fourth state, it may switch the fourth state to the first state. The control unit 400 may repeat switching the state from the first state to the fourth state. In this manner, the control unit 400 may switch between the first state and the third state by controlling the plurality of open or close valves 140, the rising of temperature of the first adsorbent 110, and the rising of temperature of the second adsorbent 210.

The control unit 400 may use various parameters as a timing for switching each process. For example, the control unit 400 may switch each process by time or switch each process by the monitored temperature. Further, the control unit 400 may switch each process by a monitored concentration of carbon dioxide of a predetermined member, and switch each process by the flow rate of the air flowing into the first inflow port 111 and the second inflow port 211.

The control unit 400 opens the first inflow valve 141, the first discharge valve 142, and the second introduction valve 243 in the first state. On the other hand, the control unit 400 closes the first introduction valve 143, the first desorption valve 145, the second inflow valve 241, the second discharge valve 242, and the second desorption valve 245 in the first state. Accordingly, in the first state, the outside air that has passed through the first apparatus 100 in such a way that at least a part of carbon dioxide of the outside air is adsorbed by the first adsorbent 110 passes through the second apparatus 200 so as to cool the second adsorbent 210 via the exhaust path 300. For example, the outside air that has passed through the inside 121 of the cylindrical vessel 120 in the first apparatus 100 passes through the outer periphery 122 of the cylindrical vessel 120 in the second apparatus 200 via the first exhaust path 301.

The control unit 400 opens the first desorption valve 145 in the second state. On the other hand, the control unit 400 closes the first inflow valve 141, the first discharge valve 142, the first introduction valve 143, the second inflow valve 241, the second discharge valve 242, the second introduction valve 243, and the second desorption valve 245 in the second state. Further, the control unit 400 raises the temperature of the first adsorbent 110. Accordingly, in the second state, the control unit 400 raises the temperature of the first adsorbent 110, causes carbon dioxide to be desorbed from the first adsorbent 110, and releases carbon dioxide to the outside.

The control unit 400 opens the second inflow valve 241, the second discharge valve 242, and the first introduction valve 143 in the third state. On the other hand, the control unit 400 closes the second introduction valve 243, the second desorption valve 245, the first inflow valve 141, the first discharge valve 142 and the first desorption valve 145 in the third state. Accordingly, in the third state, the outside air that has passed through the second apparatus 200 in such a way that at least a part of carbon dioxide of the outside air is adsorbed by the second adsorbent 210 passes through the first apparatus 100 via the exhaust path 300 in such a way that it cools the first adsorbent 110. For example, the outside air that has passed through the inside 121 of the cylindrical vessel 120 in the second apparatus 200 passes through the outer periphery 122 of the cylindrical vessel 120 in the first apparatus 100 via the second exhaust path 302.

The control unit 400 opens the second desorption valve 245 in the fourth state. On the other hand, the control unit 400 closes the first inflow valve 141, the first discharge valve 142, the first introduction valve 143, the first desorption valve 145, the second inflow valve 241, the second discharge valve 242, and the second introduction valve 243 in the fourth state. Further, the control unit 400 raises the temperature of the second adsorbent 210. Accordingly, in the fourth state, the control unit 400 raises the temperature of the second adsorbent 210, causes carbon dioxide to be desorbed from the second adsorbent 210, and releases carbon dioxide to the outside.

### <Carbon Dioxide Capture Method>

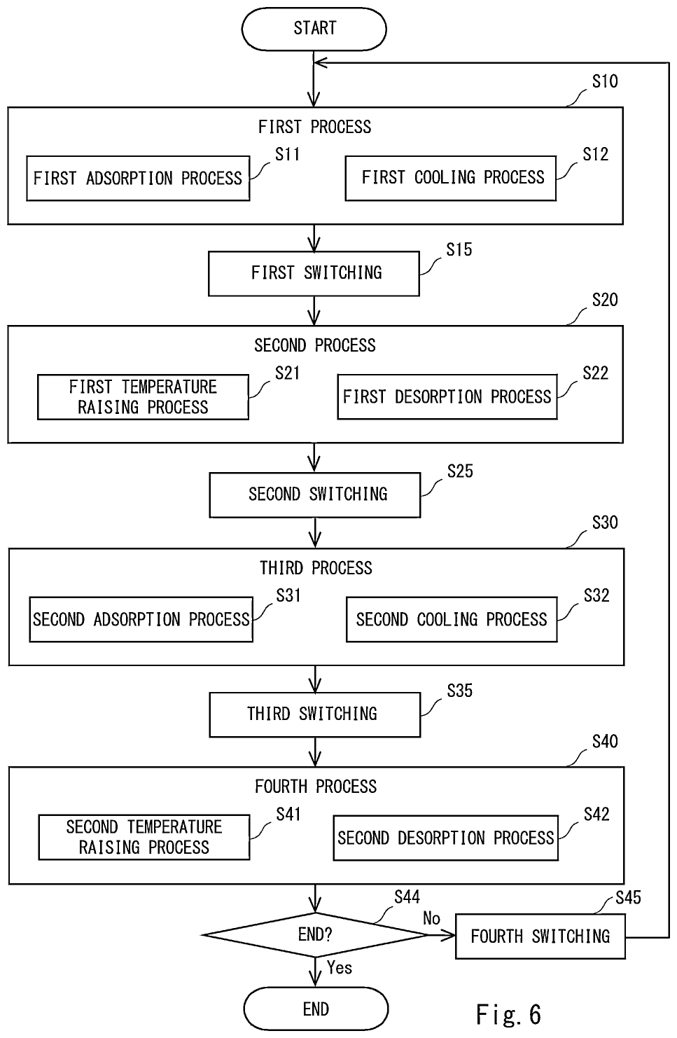

Next, a carbon dioxide capture method that uses the carbon dioxide capture apparatus 1 according to this embodiment will be described. FIG. 6 is a flowchart illustrating a carbon dioxide capture method using the carbon dioxide capture apparatus 1 according to the first embodiment. As shown in FIG. 6, the carbon dioxide capture method according to this embodiment includes a first process (Step S10), a first switching process (Step S15), a second process (Step S20), a second switching process (Step S25), a third process (Step S30), a third switching process (Step S35), a fourth process (Step S40), a determination process (Step S44), and a fourth switching process (Step S45).

### <First Process>

As shown in FIGS. 1 and 6, the first process includes a first adsorption process (Step S11) and a first cooling process (Step S12). The first adsorption process causes the outside air to be passed through the first apparatus 100 in such a way that at least a part of carbon dioxide is adsorbed by the first adsorbent 110. In the first adsorption process, at least a part of carbon dioxide of the outside air that has passed through the inside 121 of the cylindrical vessel 120 may be adsorbed. The first cooling process causes the outside air to be passed through the second apparatus 200 via the exhaust path 300 in such a way that the outside air that has passed through the first apparatus 100 in the first adsorption process cools the second adsorbent 210. In the first cooling process, the outside air that has passed through the outer periphery 122 of the cylindrical vessel 120 cools the second adsorbent 210.

The first switching process switches the first process to the second process by controlling the plurality of open or close valves 140 and rising of temperature. For example, the control unit 400 opens the first desorption valve 145. On the other hand, the control unit 400 closes the first inflow valve 141, the first discharge valve 142, the first introduction valve 143, the second inflow valve 241, the second discharge valve 242, the second introduction valve 243, and the second desorption valve 245. Further, the control unit 400 transmits an operation signal in such a way that the temperature of the first adsorbent 110 is raised. In this manner, the control unit 400 switches the first process to the second process.

### <Second Process>

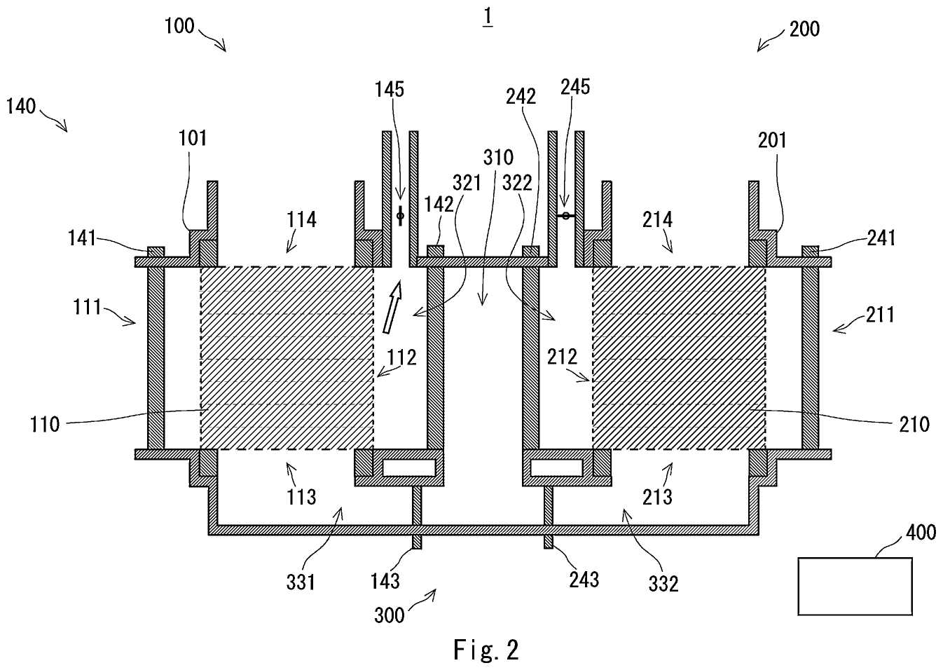

As shown in FIGS. 2 and 6, the second process includes a first temperature raising process (Step S21) and a first desorption process (Step S22). The first temperature raising process raises the temperature of the first adsorbent 110. Accordingly, carbon dioxide can be made to desorb from the first adsorbent 110. The first desorption process causes carbon dioxide to be desorbed from the first adsorbent 110 and releases carbon dioxide to the outside.

The second switching process switches the second process to the third process by controlling the plurality of open or close valves 140 and rising of temperature. For example, the control unit 400 opens the second inflow valve 241, the second discharge valve 242, and the first introduction valve 143. On the other hand, the control unit 400 closes the second introduction valve 243, the second desorption valve 245, the first inflow valve 141, the first discharge valve 142, and the first desorption valve 145. Further, the control unit 400 transmits an operation signal so as to stop heating the first adsorbent 110. In this manner, the control unit 400 switches the second process to the third process.

### <Third Process>

As shown in FIGS. 3 and 6, the third process includes a second adsorption process (Step S31) and a second cooling process (Step S32). The third adsorption process causes the outside air to be passed through the second apparatus 200 in such a way that at least a part of carbon dioxide is adsorbed by the second adsorbent 210. In the second adsorption process, at least a part of carbon dioxide of the outside air that has passed through the inside 121 of the cylindrical vessel 120 may be adsorbed. The second cooling process causes the outside air to be passed through the first apparatus 100 via the exhaust path 300 in such a way that the outside air that has passed through the second apparatus 200 in the second adsorption process cools the first adsorbent 110. In the second cooling process, the outside air that has passed through the outer periphery 122 of the cylindrical vessel 120 cools the first adsorbent 110.

The third switching process switches the third process to the fourth process by controlling the plurality of open or close valves 140 and rising of temperature. The control unit 400 opens, for example, the second desorption valve 245. On the other hand, the control unit 400 closes the first inflow valve 141, the first discharge valve 142, the first introduction valve 143, the first desorption valve 145, the second inflow valve 241, the second discharge valve 242, and the second introduction valve 243. Further, the control unit 400 transmits an operation signal in such a way that the temperature of the second adsorbent 210 is raised. In this manner, the control unit 400 switches the third process to the fourth process.

### <Fourth Process>

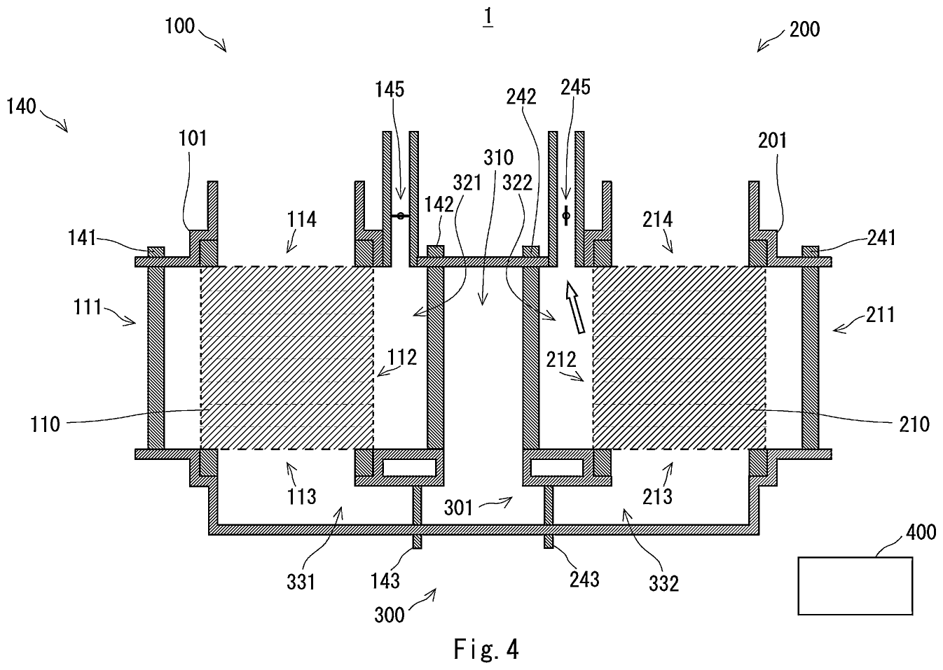

As shown in FIGS. 4 and 6, the fourth process includes a second temperature raising process (Step S41) and a second desorption process (Step S42). The second temperature raising process raises the temperature of the second adsorbent 210. Accordingly, carbon dioxide can be desorbed from the second adsorbent 210. The second desorption process causes carbon dioxide to be desorbed from the second adsorbent 210 and releases carbon dioxide to the outside.

The determination process (Step S44) determines whether or not to end the processing. When it is determined that the processing is ended (Yes), the processing is ended. On the other hand, when it is not determined that the processing is ended (No), the process proceeds to a fourth switching process.

The fourth switching process switches the fourth process to the first process. For example, the control unit 400 opens the first inflow valve 141, the first discharge valve 142, and the second introduction valve 243. On the other hand, the control unit 400 closes the first introduction valve 143, the first desorption valve 145, the second inflow valve 241, the second discharge valve 242, and the second desorption valve 245. Further, the control unit 400 transmits an operation signal so as to stop heating the second adsorbent 210. In this manner, the control unit 400 switches the fourth process to the first process.

As described above, the carbon dioxide capture method according to this embodiment may repeat the first process to the fourth process. Roughly speaking, the carbon dioxide capture method may switch between the first adsorption process and the first cooling process in the first process and the second adsorption process and the second cooling process in the third process by controlling the plurality of open or close valves 140 and rising of temperature.

Next, effects of this embodiment will be described. The carbon dioxide capture apparatus 1 according to this embodiment can use the outside air used to adsorb carbon dioxide by the first adsorbent 110 to cool the second adsorbent 210. This does not require any new motive power so as to flow refrigerant, whereby it is possible to improve cooling efficiency.

For example, Patent Literature 1 discloses a carbon dioxide capture apparatus in which a plurality of board-type adsorption plates that adsorb carbon dioxide contained in outside air are stacked at intervals therebetween. When the outside air passes between the adsorption plates, carbon dioxide of the outside air is adsorbed. In Patent Literature 1, a plurality of pipes that are penetrated through the above adsorption plate in the stacking direction are provided. Each of these pipes serves as a heat medium flow passage through which the heat medium flows, whereby the temperature of the adsorption plates is controlled. In Patent Literature 1, a pump for causing a medium to flow therethrough to cool the adsorption plates to a temperature suitable for adsorption is needed, which causes an increase in the carbon dioxide capture energy.

On the other hand, in the carbon dioxide capture apparatus 1 according to this embodiment, no new motive power to cause a refrigerant to flow is needed, whereby it is possible to improve cooling efficiency.

Further, the carbon dioxide capture apparatus 1 according to this embodiment includes the exhaust path 300 that communicates the first apparatus 100 with the second apparatus 200. Then, by controlling the open or close valves 140, the outside air that is used for adsorption in one of the first adsorbent 110 and the second adsorbent 210 can be used to cool the other one. Therefore, both adsorbents can be cooled by each other's outside air, whereby it is possible to further improve cooling efficiency.

The first adsorbent 110 and the second adsorbent 210 may be each provided in the inside 121 of the tubular cylindrical vessel 120. In this case, at least a part of carbon dioxide of the outside air that has passed through the inside 121 of the cylindrical vessel 120 is adsorbed, and the outside air that has passed through the outer periphery 122 of the cylindrical vessel 120 cools the adsorbent. Since the outside air to be cooled does not contact the adsorbent whose temperature is high, it is possible to suppress the reaction and suppress degradation of the adsorbents.

Since the first exhaust path 301 and the second exhaust path 302 are communicated with the first adsorbent 110 and the second adsorbent 210 from directions different from each other, the adsorption process and the cooling process can be separated from each other and it is thus possible to improve cooling efficiency.

Since the exhaust path is formed near the cylindrical vessel 120 including the first adsorbent 110 and the second adsorbent 210, it can be insulated by air and thus heat loss can be reduced.

### Second Embodiment

In the above-described first embodiment, by disposing a fan outside the first inflow valve 141 and outside the second inflow valve 241, a flow of causing the outside air to be processed to be introduced into the carbon dioxide capture apparatus 1 is formed. In this embodiment, a fan is disposed in the outside of the first release port 114 and the second release port 214, whereby a flow of releasing the outside air introduced into the carbon dioxide capture apparatus 1 to the outside may be formed. With this configuration as well, effects similar to those obtained in the first embodiment can be obtained.

Note that the present disclosure is not limited to the above-described embodiments and may be changed as appropriate without departing from the spirit of the present disclosure.

The present disclosure can implement each processing in the carbon dioxide capture method by causing a computer to execute the above processing as a computer program (e.g., carbon dioxide capture program).

In the above-described embodiments, the control unit 400 is formed of a computer system including a personal computer or a word processor. However, this is merely one example, and the computer may be formed of a server of a LAN, a host of computer (personal computer) communication, a computer system connected on the Internet, or the like. Further, functions may be distributed to the respective devices on the network and the entire network may form the computer.

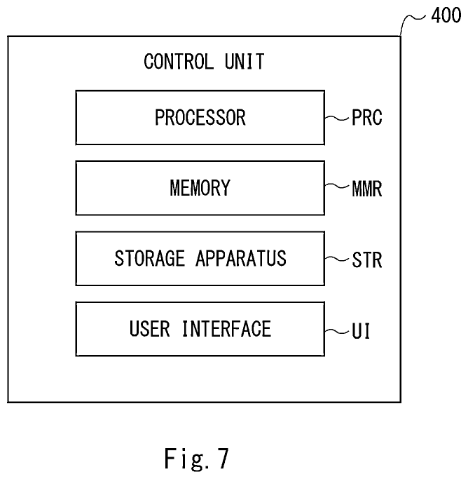

FIG. 7 is a block diagram illustrating the control unit 400 including a computer in the carbon dioxide capture apparatus 1 according to each of the first and second embodiments. As shown in FIG. 7, the control unit 400 may further include a processor PRC, a memory MMR, a storage apparatus STR, and a user interface UI. The storage apparatus STR stores processing executed by the control unit 400 in a form of a program. Further, the processor PRC causes the program to be loaded into the memory MMR from the storage apparatus STR, thereby executing this program. Accordingly, the processor PRC implements the function of the control unit 400. The user interface UI may include an input device such as a keyboard, a mouse, or an image-capturing apparatus, and an output device such as a display, a printer, or a speaker.

The control unit 400 may be implemented by special-purpose hardware. Further, some or all of the components of the control unit 400 may each be implemented by a general-purpose or special-purpose circuitry, a processor PRC, or a combination of them. They may be configured using a single chip, or a plurality of chips connected through a bus. Some or all of the components of the control unit 400 may be implemented by a combination of the above-described circuitry, etc. and a program. Further, a Central Processing Unit (CPU), a Graphics Processing Unit (GPU), a Field-Programmable Gate Array (FPGA), a quantum processor (quantum computer control chip) and so on may be used as the processor PRC.

Further, when some or all of the components of the control unit 400 are implemented by a plurality of information processing apparatuses, circuits, or the like, the plurality of information processing apparatuses, the circuits, or the like may be disposed in one place in a centralized manner or arranged in a distributed manner. For example, the information processing apparatuses, the circuits, and the like may be implemented as a form such as a client-server system, a cloud computing system or the like in which they are connected to each other through a communication network NW. Further, the functions of the control unit 400 may be provided in the form of Software as a Service (Saas).

The following carbon dioxide capture method and carbon dioxide capture program are also included within a technical idea of this embodiment.

### (Supplementary Note 1)

A carbon dioxide capture method performed using a carbon dioxide capture apparatus comprising:

a first apparatus including a first adsorbent that adsorbs at least a part of carbon dioxide contained in outside air to be processed;

a second apparatus including a second adsorbent that adsorbs at least a part of the carbon dioxide contained in the outside air; and

an exhaust path configured to communicate the first apparatus with the second apparatus, the carbon dioxide capture method comprising:

a first adsorption process for causing the outside air to be passed through the first apparatus in such a way that at least a part of the carbon dioxide is adsorbed by the first adsorbent; and a first cooling process for causing the outside air to pass through the second apparatus via the exhaust path in such a way that the outside air that has passed through the first apparatus in the first adsorption process cools the second adsorbent.

### Supplementary Note 2

The carbon dioxide capture method according to Supplementary Note 1, wherein
the first adsorbent and the second adsorbent are each disposed inside a tubular cylindrical vessel,
the first apparatus comprises one or a plurality of the cylindrical vessels including the first adsorbent,
the second apparatus comprises one or a plurality of the cylindrical vessels including the second adsorbent,
in the first adsorption process,
at least a part of the carbon dioxide of the outside air that has passed through the inside of the cylindrical vessel is adsorbed, and
in the first cooling process,
the outside air that has passed through an outer periphery of the cylindrical vessel cools the second adsorbent.

### Supplementary Note 3

The carbon dioxide capture method according to Supplementary Note 1, wherein
the carbon dioxide capture apparatus further comprises:
a plurality of open or close valves disposed in the first apparatus, the second apparatus, and the exhaust path; and
a control unit configured to control the plurality of open or close valves, rising of temperature of the first adsorbent, and rising of temperature of the second adsorbent,
the carbon dioxide capture method further comprises a first switching process for switching a first process to a second process by controlling the plurality of open or close valves and the rising of temperature,
the first process comprises the first adsorption process and the first cooling process,
the second process comprises:
   a first temperature raising process for raising the temperature of the first adsorbent; and
   a first desorption process for causing the carbon dioxide to be desorbed from the first adsorbent and releasing the carbon dioxide to the outside.

### Supplementary Note 4

The carbon dioxide capture method according to Supplementary Note 1, wherein
the carbon dioxide capture apparatus further comprises:
   a plurality of open or close valves disposed in the first apparatus, the second apparatus, and the exhaust path; and
   a control unit configured to control the plurality of open or close valves, rising of temperature of the first adsorbent, and rising of temperature of the second adsorbent,
the carbon dioxide capture method further comprises a process for switching a first process and a third process by controlling the plurality of open or close valves and the rising of temperature,
the first process comprises the first adsorption process and the first cooling process,
the third process comprises:
   a second adsorption process for causing the outside air to pass through the second apparatus in such a way that at least a part of the carbon dioxide is adsorbed by the second adsorbent; and
   a second cooling process for causing the outside air to pass through the first apparatus via the exhaust path in such a way that the outside air that has passed through the second apparatus in the second adsorption process cools the first adsorbent.

### Supplementary Note 5

The carbon dioxide capture method according to Supplementary Note 4, further comprising:
a first switching process for switching the first process to a second process;
a second switching process for switching the second process to the third process;
a third switching process for switching the third process to a fourth process; and
a fourth switching process for switching the fourth process to the first process, wherein
the second process comprises:
   a first temperature raising process for raising the temperature of the first adsorbent; and
   a first desorption process for causing the carbon dioxide to be desorbed from the first adsorbent and releasing the carbon dioxide to the outside, and
the fourth process comprises:
   a second temperature raising process for raising the temperature of the second adsorbent; and
   a second desorption process for causing the carbon dioxide to be desorbed from the second adsorbent and releasing the carbon dioxide to the outside.

### Supplementary Note 6

A carbon dioxide capture program executed using a carbon dioxide capture apparatus comprising:
a first apparatus including a first adsorbent that adsorbs at least a part of carbon dioxide contained in outside air to be processed;
a second apparatus including a second adsorbent that adsorbs at least a part of the carbon dioxide contained in the outside air; and
an exhaust path configured to communicate the first apparatus with the second apparatus;
a plurality of open or close valves disposed in the first apparatus, the second apparatus, and the exhaust path; and
a control unit configured to control the plurality of open or close valves, rising of temperature of the first adsorbent, and rising of temperature of the second adsorbent,
the program causing a computer to execute:
   a first adsorption process for causing the outside air to be passed through the first apparatus in such a way that at least a part of the carbon dioxide is adsorbed by the first adsorbent; and
   a first cooling process for causing the outside air to pass through the second apparatus via the exhaust path in such a way that the outside air that has passed through the first apparatus in the first adsorption process cools the second adsorbent.

### Supplementary Note 7

The carbon dioxide capture program according to Supplementary Note 6, wherein
the first adsorbent and the second adsorbent are each disposed inside a tubular cylindrical vessel,
the first apparatus comprises one or a plurality of the cylindrical vessels including the first adsorbent,
the second apparatus comprises one or a plurality of the cylindrical vessels including the second adsorbent,
in the first adsorption process,
at least a part of the carbon dioxide of the outside air that has passed through the inside of the cylindrical vessel is adsorbed, and in the first cooling process,
the outside air that has passed through an outer periphery of the cylindrical vessel cools the second adsorbent.

### Supplementary Note 8

The carbon dioxide capture program according to Supplementary Note 6, wherein
the program causes a computer to further execute a first switching process for switching a first process to a second process by controlling the plurality of open or close valves and the rising of temperature,
the first process comprises the first adsorption process and the first cooling process,
the second process comprises:
   a first temperature raising process for raising the temperature of the first adsorbent; and
   a first desorption process for causing the carbon dioxide to be desorbed from the first adsorbent and releasing the carbon dioxide to the outside.

### Supplementary Note 9

The carbon dioxide capture program according to Supplementary Note 6, wherein
the program causes a computer to further execute a process for switching a first process and a third process by controlling the plurality of open or close valves and the rising of temperature,
the first process comprises the first adsorption process and the first cooling process, and
the third process comprises:
   a second adsorption process for causing the outside air to pass through the second apparatus in such a way that at least a part of the carbon dioxide is adsorbed by the second adsorbent; and
   a second cooling process for causing the outside air to pass through the first apparatus via the exhaust path in such a way that the outside air that has passed through the second apparatus in the second adsorption process cools the first adsorbent.

### Supplementary Note 10

The carbon dioxide capture program according to Supplementary Note 9, wherein
the program causes a computer to further execute:
   a first switching process for switching the first process to a second process;
   a second switching process for switching the second process to the third process;
   a third switching process for switching the third process to a fourth process; and
   a fourth switching process for switching the fourth process to the first process,
the second process comprises:
   a first temperature raising process for raising the temperature of the first adsorbent; and
   a first desorption process for causing the carbon dioxide to be desorbed from the first adsorbent and releasing the carbon dioxide to the outside, and
the fourth process comprises:
   a second temperature raising process for raising the temperature of the second adsorbent; and
   a second desorption process for causing the carbon dioxide to be desorbed from the second adsorbent and releasing the carbon dioxide to the outside.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A carbon dioxide capture apparatus comprising:
a first apparatus including a first adsorbent that adsorbs at least a part of carbon dioxide contained in outside air to be processed;
a second apparatus including a second adsorbent that adsorbs at least a part of the carbon dioxide contained in the outside air; and
an exhaust path configured to communicate the first apparatus with the second apparatus,
wherein the outside air that has passed through the first apparatus in such a way that at least a part of the carbon dioxide is adsorbed by the first adsorbent passes through the second apparatus via the exhaust path so as to cool the second adsorbent.

2. The carbon dioxide capture apparatus according to claim 1, wherein
the first adsorbent and the second adsorbent are each disposed inside a tubular cylindrical vessel,
the first apparatus comprises one or a plurality of the cylindrical vessels including the first adsorbent,
the second apparatus comprises one or a plurality of the cylindrical vessels including the second adsorbent,
at least a part of the carbon dioxide of the outside air that has passed through the inside of the cylindrical vessel is adsorbed, and
the outside air that has passed through an outer periphery of the cylindrical vessel cools the second adsorbent.

3. The carbon dioxide capture apparatus according to claim 2, wherein
the first apparatus comprises:
a first inflow port through which the outside air passing through the inside of the cylindrical vessel flows in;
a first discharge port from which the outside air that has passed through the inside of the cylindrical vessel is discharged;
a first introduction port through which the outside air passing through an outer periphery of the cylindrical vessel is introduced; and
a first release port from which the outside air that has passed through the outer periphery of the cylindrical vessel is released, and
the second apparatus comprises:
a second inflow port through which the outside air passing through the inside of the cylindrical vessel flows in;
a second discharge port through which the outside air that has passed through the inside of the cylindrical vessel is discharged;
a second introduction port through which the outside air passing through an outer periphery of the cylindrical vessel is introduced; and
a second release port through which the outside air that has passed through an outer periphery of the cylindrical vessel is released.

4. The carbon dioxide capture apparatus according to claim 1, further comprising:
a plurality of open or close valves disposed in the first apparatus, the second apparatus, and the exhaust path; and
a control unit configured to switch a first state to a second state by controlling the plurality of open or close valves, rising of temperature of the first adsorbent, and rising of temperature of the second adsorbent, wherein
in the first state,
the outside air that has passed through the first apparatus in such a way that at least a part of the carbon dioxide is adsorbed by the first adsorbent passes through the second apparatus via the exhaust path so as to cool the second adsorbent, and
in the second state,
the temperature of the first adsorbent is raised, the carbon dioxide is caused to be desorbed from the first adsorbent, and the carbon dioxide is released to the outside.

5. The carbon dioxide capture apparatus according to claim 1, further comprising:
a plurality of open or close valves disposed in the first apparatus, the second apparatus, and the exhaust path; and
a control unit configured to switch between a first state and a third state by controlling the plurality of open or close valves, rising of temperature of the first adsorbent, and rising of temperature of the second adsorbent, wherein
in the first state,
the outside air that has passed through the first apparatus in such a way that at least a part of the carbon dioxide is adsorbed by the first adsorbent passes through the second apparatus via the exhaust path so as to cool the second adsorbent, and
in the third state,
the outside air that has passed through the second apparatus in such a way that at least a part of the carbon dioxide is adsorbed by the second adsorbent passes through the first apparatus via the exhaust path so as to cool the first adsorbent.

6. The carbon dioxide capture apparatus according to claim 5, wherein
after the control unit switches the first state to a second state, it switches the second state to the third state,
after the control unit switches the third state to a fourth state, it switches the fourth state to the first state,
in the second state,
the temperature of the first adsorbent is raised, the carbon dioxide is caused to be desorbed from the first adsorbent, and the carbon dioxide is released to the outside, and
in the fourth state,
the temperature of the second adsorbent is raised, the carbon dioxide is caused to be desorbed from the second adsorbent, and the carbon dioxide is released to the outside.

7. The carbon dioxide capture apparatus according to claim 5, wherein
the exhaust path comprises:
a first exhaust path through which the outside air flows from the first apparatus to the second apparatus; and
a second exhaust path through which the outside air flows from the second apparatus to the first apparatus, and
the first exhaust path and the second exhaust path include parts communicated with the first apparatus and the second apparatus from directions different from each other.

8. The carbon dioxide capture apparatus according to claim 7, wherein
the first apparatus comprises:
a first inflow port through which the outside air flows in;
a first discharge port through which the outside air flowing in through the first inflow port is discharged;
a first introduction port through which the outside air discharged from the second apparatus is introduced; and
a first release port through which the outside air introduced from the first introduction port is released to the outside,
the second apparatus comprises:
a second inflow port through which the outside air flows in;
a second discharge port through which the outside air flowing in through the second inflow port is discharged;
a second introduction port through which the outside air discharged from the first apparatus is introduced; and
a second release port through which the outside air introduced from the second introduction port is released to the outside,
the exhaust path comprises:
a common space disposed between the first apparatus and the second apparatus;
a first discharge space disposed between the common space and the first discharge port;
a first introduction space disposed between the common space and the first introduction port;
a second discharge space disposed between the common space and the second discharge port; and
a second introduction space disposed between the common space and the second introduction port,
the plurality of open or close valves comprise:
a first inflow valve that opens or closes the first inflow port;
a first discharge valve that opens or closes a part between the common space and the first discharge space;
a first introduction valve that opens or closes a part between the common space and the first introduction space;
a second inflow valve that opens or closes the second inflow port;
a second discharge valve that opens or closes a part between the common space and the second discharge space; and
a second introduction valve that opens or closes a part between the common space and the second introduction space,
the first discharge valve, the first introduction valve, the second discharge valve, and the second introduction valve are disposed in the exhaust path,
the first exhaust path includes the first discharge space, the common space, and the second introduction space that are communicated with each other, and
the second exhaust path includes the second discharge space, the common space, and the first introduction space that are communicated with each other.

9. The carbon dioxide capture apparatus according to claim 8, further comprising:
a first desorption valve that opens or closes a part between the first discharge space and the outside; and
a second desorption valve that opens or closes a part between the second discharge space and the outside.

10. The carbon dioxide capture apparatus according to claim 7, wherein
a release port that releases the outside air to the outside is disposed on the side of the introduction port opposite to the side thereof that communicates with the first exhaust path in the second apparatus, and
an inflow port through which the outside air flows in from the outside is disposed on the side of the discharge port opposite to the side thereof that communicates with the second exhaust path in the second apparatus.

11. A carbon dioxide capture method performed using a carbon dioxide capture apparatus comprising:
a first apparatus including a first adsorbent that adsorbs at least a part of carbon dioxide contained in outside air to be processed;
a second apparatus including a second adsorbent that adsorbs at least a part of the carbon dioxide contained in the outside air; and
an exhaust path configured to communicate the first apparatus with the second apparatus, the carbon dioxide capture method comprising:
a first adsorption process for causing the outside air to be passed through the first apparatus in such a way that at least a part of the carbon dioxide is adsorbed by the first adsorbent; and
a first cooling process for causing the outside air to pass through the second apparatus via the exhaust path in such a way that the outside air that has passed through the first apparatus in the first adsorption process cools the second adsorbent.

12. A non-transitory computer readable medium storing a carbon dioxide capture program executed using a carbon dioxide capture apparatus comprising:
a first apparatus including a first adsorbent that adsorbs at least a part of carbon dioxide contained in outside air to be processed;
a second apparatus including a second adsorbent that adsorbs at least a part of the carbon dioxide contained in the outside air; and
an exhaust path configured to communicate the first apparatus with the second apparatus;
a plurality of open or close valves disposed in the first apparatus, the second apparatus, and the exhaust path; and
a control unit configured to control the plurality of open or close valves, rising of temperature of the first adsorbent, and rising of temperature of the second adsorbent,
the program causing a computer to execute:
a first adsorption process for causing the outside air to be passed through the first apparatus in such a way that at least a part of the carbon dioxide is adsorbed by the first adsorbent; and
a first cooling process for causing the outside air to pass through the second apparatus via the exhaust path in such a way that the outside air that has passed through the first apparatus in the first adsorption process cools the second adsorbent.
